(19) United States
(12) Patent Application Publication
(10) Pub. No.: US 2004/0127396 A1
(43) Pub. Date: Jul. 1, 2004

(54) Use of furin and furin-like protease inhibitors in the treatment of inflammatory or matrix remodelling diseases

(76) Inventor: Claire Dubois, Sherbrooke (CA)

Correspondence Address: Stephen P. Georgiev, SMART & BIGGAR, Suite 3400, 1000 de la Gauchetiere Street West, Montreal, QC, H3B 4W5, CA

(21) Appl. No.: 09/885,914

(22) Filed: Jun. 22, 2001

Related U.S. Application Data
(60) Provisional application No. 60/213,995, filed on Jun. 26, 2000.

(30) Foreign Application Priority Data
Jun. 23, 2000 (CA) 2,312,109

(51) Int. Cl. A61K 38/17

(52) U.S. Cl. 514/2

(57) ABSTRACT

The present invention provides methods, uses and compositions of an α1-antitrypsin variant called PDX or a construct, variant, analog, peptide, peptidomimetic, salt, complex or derivative thereof for the treatment of inflammatory or erosive diseases such as rheumatoid arthritis.

This application claims the benefit of U.S. Provisional Application No. 60/213,995 filed Jun. 26, 2000.

### FIELD OF THE INVENTION

The present invention relates to the treatment of diseases related to inflammatory or matrix remodelling conditions in mammals.

### BACKGROUND OF THE INVENTION

Rheumatoid arthritis (RA) comprises a group of autoimmune diseases afflicting 1% of the population and leading to significant morbidity. There are no known cures for RA or other inflammatory autoimmune arthropathies. Arthritis is a multifactorial disease for which single therapies have so far yielded disappointing results. In order to address this complex problem, several strategies are possible such as treating the disease with a combinatorial approach or by finding a unique biological target with a large activity spectrum. Rheumatoid arthritis is characterized by chronic inflammation in joints and concomitant destruction of cartilage and bones. In the joint, three aspects are important, including synovial cell hyperproliferation/activation, inflammation and matrix destruction. Many of these events are mediated by bioactive proteins; among them are a number of cytokines, growth factors and matrix metalloproteinases.

Several biologically active peptides and proteins are synthesized initially as larger and inactive precursor proteins that are endoproteolytically cleaved to generate the regulatory protein in a mature and biologically active form. These include hormones, neuropeptides, enzymes, growth factors, cell-surface receptors and viral envelope glycoproteins. In many of these precursors, endoproteolysis takes place after a sequence of two or more basic residues (K or R). Seven closely related mammalian subtilisin/kexin-like serine proteinases with this cleaving specificity have been discovered in recent years (references 1 and 2). They have been grouped under the generic name of proprotein convertases (PC) and named furin, PC1/PC3, PC2, PC4, PACE4, PC5/PC6A, PC5/PC6B and PC7/PC8. The convertase furin was the first to be discovered and is considered the prototype of the PC family.

Furin is localized mainly in trans-Golgi network (TGN) (references 3 and 4) and can also translocate between the cell surface and the TGN. Substrate specificity studies have revealed that furin requires an R-X-X-R motif for cleavage while the R-X-K/R-R sequence provides an optimum processing site (reference 5). According to their tissue distribution, the proprotein convertases can be classified into distinct subgroups where furin and PC7/PC8 are ubiquitously distributed, PACE4, PC5/PC6A and PC5/PC6B are expressed to varying degrees in many tissues and whereas the other convertases PC1, PC2 and PC4 are restricted to specific tissues such as neural and endocrine ones (PC1, PC2) and testicular spermatogenic cells (PC4).

A common structural feature of some polypeptide growth involved in growth and inflammation is the presence of basic residues at the proteolytic processing site within the molecule. This is the case of PDGF A-chain and B-chain (references 6 and 7) and most members of the extended family of TGF β (reference 8). The PC-like site in most of these precursors corresponds to the R-X-K/R-R consensus cleavage of furin and in some case PACE4 and PCS/6 or PC7 (reference 9). Dubois et al. have shown by in vitro and in vivo studies that TGF β1 is efficiently processed by furin releasing the genuine mature growth factor (reference 10). PDGF is expressed by synoviocytes or macrophages in rheumatoid arthritis (RA) and are major contributors to synovial hyperplasia and pannus formation by stimulation of DNA synthesis in synovial fibroblast (references 11 and 12). PDGF is also able to sustain cartilage degradation presumably due to its ability to stimulate collagenase synthesis and neovascularization (references 12-15).

Other factors, such as transforming growth factor beta (TGF β), may be stimulatory or inhibitory depending on the cell type and local concentration. TGF β is present in the synovial fluid and synovial cell cultures in RA. When applied intraarticularly, this factor has been shown to cause synovitis through its ability to induce neutrophil recruitment, synovial hyperplasia and loss of cartilage proteoglycans (references 16-20). In contrast, TGF β when administered systemically inhibits acute and chronic symptoms of streptococcal cell wall-induced arthritis in rats through its immunosuppressive effects (reference 21).

Beside these growth factors, metalloproteinases are other contributors in the pathogenesis of RA. Among the metalloproteinases, both the TNF α-converting enzymes TACE (MMP-17), aggrecenase (ADAMTS11, ADAMTS4) MDC-9, and members of the MT-MMP family were found to be first synthesized as inactive precursor molecules (references 22-29). Their amino acid sequences at the activation cleavage site correspond to the consensus furin recognition sequence. The contribution of furin or furin MDC-9 activation has been recently documented (reference 23). The removal of the TACE prodomain in a late Golgi compartment has also been documented (reference 28). Clarke et al. disclosed that human TACE was processed in yeast at the RVKR⇓R site (reference 29). For gelatinase A, the contribution of furin-activated MT1-MMP in its activation is also documented (references 30 and 31).

TACE, through the activation of TNF α, plays a major and well-documented contribution in the chronic inflammatory aspect of RA. In animal models and clinical trials, blockage of this cytokine using neutralizing antibodies has shown impressive anti-inflammatory effect with dramatic reduction of acute phase protein detection in the majority of patients and an influence on the erosive aspect of the pathology (references 32-34). Therefore, TACE represents an important clinical target for therapeutic intervention in a variety of inflammatory diseases including RA.

Recent findings indicate that gelatinase A is expressed in the normal synovial lining and in its pathological extension, the pannocytes of the invasive pannus tissue (reference 35). This way, gelatinase A may participate in the remodelling of the normal lining and its pathological extension, a tissue deeply involved in matrix destruction in arthritis.

Destruction of the cartilage matrix protein aggrecan is one of the early hallmarks of arthritis. Depletion of aggrecan from cartilage compromises the weight-bearing properties of the tissue and lead to further mechanical disruption of the cartilage. Recently, a new metalloprotease responsible for aggrecan cleavage (aggrecanase) was isolated from bovine cartilage. This protease is a member of the ADAM (a disintegrin and metalloproteinase) family that cleaves aggrecan at the Glu³⁷³-Ala³⁷⁴ bond (reference 24) resulting in the production of aggrecan fragments indistinguishable from those found in arthropathies (reference 36). Since then, another ADAM family member with aggrecanase activity has been cloned and called aggrecanase 2 or ADAMTS-11 (reference 25).

Protein-based serine protease inhibitors have been evaluated to block furin activity. The most specific one is an engineered variant of the endogenous elastase inhibitor, the serpin α₁-antitrypsin (α₁-AT). To engineer this derivative, Anderson et al. have mutated the natural reactive site (Ala-Ile-Pro-Met³⁵⁸) of the serpin for an Arg-Ile-Pro-Arg³⁵⁸ sequence (reference 37). This mutant named PDX now mimics the minimum consensus sequence (R-X-X-R) required for furin recognition and has been shown to be a potent furin inhibitor in vitro and in cells (references 37 and 38). Recent studies using purified enzymes have demonstrated that PDX is a potent inhibitor of furin that also inhibits PC6 to some extent (references 39 and 40). PDX has also been shown to block furin activity in an in vitro measles virus model resulting in loss of syncitia formation (reference 41). Thus, PDX offers an interesting approach to address the role of furin in pathological conditions.

U.S. Pat. No. 6,022,855 issued on Feb. 8, 2000, herein incorporated by reference, is related to the inhibition of furin convertase with variants of the serpin α₁-antitrypsin.

### SUMMARY OF THE INVENTION

The present invention provides methods, uses and compositions for treating inflammatory or matrix remodelling diseases in mammals by inhibiting proprotein convertase activity.

Accordingly, the present invention provides a method for the treatment in a mammal of an inflammatory or erosive disease, said method comprising administering to the mammal a compound capable of inhibiting a proprotein convertase.

In an embodiment, the present invention provides methods, uses and compositions for treating inflammatory or matrix remodelling diseases in mammals by inhibiting furin or furin-like protease activity.

In certain embodiments, the present invention further provides methods, uses and compositions of a compound capable of inhibiting a proprotein convertase. In an embodiment, the proprotein convertase is furin or a furin-like protease.

Particularly, there are provided methods, uses and compositions of PDX or a construct, variant, analog, peptide, peptidomimetic, salt, complex or derivative thereof for the treatment of inflammatory or matrix remodelling diseases in mammals.

In an embodiment, the invention further provides a composition for the treatment in a mammal of an inflammatory or erosive disease, said composition comprising a compound selected from PDX or a construct, variant, analog, PDX-related peptide, PDX-related-peptidomimetic, their salts, complexes or derivatives.

The invention further provides a commercial package comprising a compound or composition of the invention together with instructions for treating inflammatory or matrix remodelling diseases in mammals.

### BRIEF DESCRIPTION OF THE DRAWINGS

FIG. 1 depicts the in vitro production of PDX in rat synoviocytes.

FIGS. 2A and 2B depict the on-off regulation of PDX and GFP expression.

FIGS. 3A and 3B show that PDX blocks the proteolytic maturation of a furin-specific substrate TGF β1 in rat synovial cells.

FIG. 4A shows that PDX blocks the production of bioactive TGF β1.

FIG. 4B shows that PDX blocks the production of bioactive PDGF by rat synovial cells.

FIG. 5A depicts the proteolytic conversion of TACE in LoVo cells.

FIG. 5B depicts the effect of Dec-RVKR-CH₂Cl on TACE maturation in LoVo cells.

FIG. 5C depicts the effect of furin on α-TNFR p75 cell surface expression.

FIG. 5D depicts the effect of Dec-RVKR-CH₂Cl on TNF α release from MonoMac-1 cells.

FIG. 5E depicts the inhibition of furin-mediated processing of endogenous TACE by PDX.

FIG. 6 depicts the inhibition of furin-mediated processing of endogenous gelatinase A (MMP-2) by PDX.

FIG. 7 depicts the inhibition of rat synovial cell growth by PDX.

FIG. 8 depicts the anti-inflammatory effect of PDX virus in a collagen-induced arthritis model.

FIG. 9 depicts the transfer vector pAdTR5F-DC-GFP derived from pAdBM5.

### DESCRIPTION OF PREFERRED EMBODIMENTS

The invention relates to the use of a compound capable of inhibiting a proprotein convertase for the treatment of inflammatory and erosive diseases. In an embodiment the invention relates to the inhibition of furin or furin-like protease activity for the treatment of inflammatory and erosive diseases. In certain embodiments the compound capable of inhibiting a proprotein convertase is PDX or a construct, variant, analog, peptide, peptidomimetic, salt, complex or derivative thereof.

In particular, the present invention relates to the use of PDX or a construct, variant, analog, peptide, peptidomimetic, salt, complex or derivative thereof for the preparation of pharmaceutical compositions or for gene therapy in systemic and intrasynovial uses of PDX related to the inhibition of furin or furin-like protease activity in inflammatory and erosive diseases. Furin-like protease activity includes the activity of proprotein convertases such as PACE4, PC5/6 or PC7. Erosive diseases are included under matrix remodelling diseases.

Preferably, the diseases are arthritis (rheumatoid arthritis, arthrosis), glomerulonephritis, pulmonary fibrosis, abnormal wound healing, degenerative cartilage loss following traumatic joint injury, inflammatory bowel disease, Cheliac diseases and of type 11 mellitus diabetis, atheriosclerosis, psoriasis and other diseases characterized by furin or furin-like protease activity. More preferably, the disease is rheumatoid arthritis.

The present invention also includes the use of PDX or a construct, variant, analog, peptide, peptidomimetic, salt, complex or derivative thereof in the treatment of diseases related to synoviocytes/, chondrocytes/and other cell types/hyperplesia including but not restricted to the articular joint.

Further provided are compounds and compositions containing furin-related/or proprotein convertases-related/cleavage sites inhibitory activities and the use of such compounds and compositions as anti-inflammatory, anti-hyperplesia and metalloprotease inhibitors (including but not restricted to TACE, gelatinase A, aggrecanase) in mammals including humans. For example, by inhibiting the ability of furin or furin-like proteases to act at the furin-related or proprotein convertases-related cleavage sites of metalloproteases, PDX or a construct, variant, analog, peptide, peptidomimetic, salt, complex or derivative thereof also inhibits the activity of the metalloproteases. Inhibiting the action of metalloproteases is therefore useful in the treatment of diseases in which metalloproteases are implicated.

In one embodiment, pharmaceutical compositions comprise constructs, variants, analogs, peptides, peptidomimetics, salts, complexes or derivatives of PDX for use in the treatment of inflammatory or matrix remodelling disorders.

In one preferred embodiment, pharmaceutical compositions are used in gene therapy methods for systemic or intraarticular delivery.

There is provided, for use in gene therapy, pharmaceutically acceptable compositions comprising a therapeutically effective amount of a gene therapy delivery system encoding PDX or a construct, variant, analog, peptide, peptidomimetic, salt, complex or derivative thereof.

Gene therapy involves the introduction of nucleic acid material into target cells of a patient. The nucleic acid material introduced generally codes for a therapeutic agent that is effective against a specific disease, particularly a disease that affects the target cells and/or surrounding tissues.

A number of techniques can be used to introduce nucleic acid material into the cells. These include:

Transfection using vectors such as viruses, particularly retroviruses, adenoviruses and adeno-associated viruses.

Biolisitics using high-speed metal particles coated with the nucleic acid material to pierce cells thus introducing the material.

Injecting liposomes into the target tissue where the liposomes encapsulate the nucleic acid material.

Using cationic lipids that aggregate with nucleic acid material and are subsequently taken up by the target cells.

Using peptides that link to the nucleic acid material as a vector rather than whole viruses.

The use of adenoviruses is particularly preferred method of gene therapy.

Gene therapy provides an advantage over existing pharmacological methods since gene therapy enables specific targeting of a therapeutic agent to a tissue or tissues. Vectors can be expressed in specific tissues and are thus useful in facilitating enhanced expression in tissues as well as in targeting expression with tissue specificity. A vector encoding a therapeutic product can be introduced into a tissue so that the tissue will express the therapeutic product.

While it is possible that PDX compounds may be administered as constructs, peptides or raw chemical entities, it is preferable to administer the active ingredient as a pharmaceutical formulation.

It will be appreciated that the amount of the compounds required for use in treatment will vary not only with the particular compound selected but also with the route of administration, the nature of the condition being treated, and the age and condition of the patient, and ultimately will be at the discretion of the attendant physician. Optimal administration rates for a given protocol of administration can be readily ascertained by those skilled in the art, using conventional dosage determination tests conducted with regard to the foregoing guidelines. See as a general guideline, Remington's Pharmaceutical Science, 16^{th} Edition, Mack (Ed.), 1980.

According to the present invention, a “therapeutically effective amount” of a pharmaceutical composition is an amount which is sufficient to achieve the desired pharmacological effect. Generally, the dosage required to provide an effective amount of the composition, and which can be adjusted by one of ordinary skill in the art, will vary, depending upon the age, health, physical condition, sex, weight and extent of disease, of the recipient. Additionally, the dosage may be determined by the frequency of treatment and the nature and scope of the desired effect. The desired dose may conveniently be presented in a single dose or as divided doses administered at appropriate intervals, for example as two, three, four or more sub-doses per day.

The compositions described herein may be administered as part of a sustained release formulation (i.e., a formulation such as a capsule or sponge that effects a slow release of modulating agent following administration). Such formulations may generally be prepared using well known technology and administered by, for example, oral, rectal or subcutaneous implantation, or by implantation at the desired target site. Sustained-release formulations may contain a modulating agent dispersed in a carrier matrix and/or contained within a reservoir surrounded by a rate controlling membrane. Carriers for use within such formulations are bio-compatible, and may also be biodegradable; preferably, the formulation provides a relatively constant level of modulating agent release. The amount of modulating agent contained within a sustained release formulation depends upon the site of implantation, the rate and expected duration of release and the nature of the condition to be treated or prevented.

The pharmaceutical composition may be administered orally (including sublingually), parenterally (including intramuscularly, intraarticularly, sub-cutaneously or intravenously), by inhalation (including spray), rectally or topically. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Compositions generally include conventional excipients; i.e., pharmaceutically acceptable organic or inorganic carrier substances that do not deleteriously react with the active compounds. Suitable pharmaceutically acceptable carriers include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethylcellulose, polyvinylpyrrolidone, etc. The pharmaceutical preparations can be sterilized and if desired, mixed with auxilliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds.

For parenteral application, particularly suitable vehicles consist of solutions, preferably oily or aqueous solutions, as well as suspensions, emulsions, or implants, including suppositories. Ampoules are convenient unit dosages.

For enteral application, particularly suitable are tablets, dragees or capsules having talc and/or a carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc, or slow-release polymers or other compounds formulated with or without inherent complementary or tissue-specific physical intervention capabilities.

Proper fluidity can be maintained, for example, by the use of a coating such as lecithin, by the maintenance of the required particle size in the case of dispersion and by the use of surfactants. Non-aqueous vehicles such as cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, availability (e.g. binding to heparan-sulfate proteoglycans at the myofiber extracellular matrix), sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. Retention of bioavailability in a tissue may be influenced by co-injection or co-administration with a stabilizing agent that would localize the invention as treatment to the fiber sarcoplasm or to the extracellular matrix as desired. Any vehicle, diluent, or additive used would have to be compatible with the compounds.

Sterile injectable solutions can be prepared by incorporating the active compounds in the required amount of the appropriate solvent with various of the other ingredients, as desired.

A pharmacological formulation can be administered to the patient in an injectable formulation containing any compatible carrier, such as various vehicle, adjuvants, additives, vectors, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include those described in: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compounds utilized can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred.

In one embodiment, the compounds of the present invention can be administered initially by intravenous injection to bring blood levels to a suitable level. The patient's levels are then maintained by an oral dosage form, although other forms of administration, dependent upon the patient's condition and as indicated above, can be used.

When desired, the above-described formulations adapted to give sustained release or pro-drugs of the active ingredient may be employed.

PDX and related-compounds may also be used in combination with other therapeutically active agents, for example, cytotoxic, corticosteroid, non-corticosteroid, immunosuppressive and antiinflammatory drugs and gene therapy agents.

The combinations referred to above may conveniently be presented for use in the form of a pharmaceutical formulation and these pharmaceutical formulations comprising a combination as defined above together with a pharmaceutically acceptable carrier comprise a further aspect of the invention. The individual components of such combinations may be administered either sequentially or simultaneously in separate or combined pharmaceutical formulations.

The following examples further illustrate the practice of this invention, but are not intended to be limiting thereof. It will be appreciated that the selection of actual amounts of PDX and related-compounds to be administered to any individual patient (human or animal) will fall within the discretion of the attending physician and will be prescribed in a manner commensurate with the appropriate dosages depending on the stage of the disease and like factors uniquely within the purview of the attending physician.

### EXAMPLE 1

A Method to Produce High Levels of PDX in Mammalian Cells

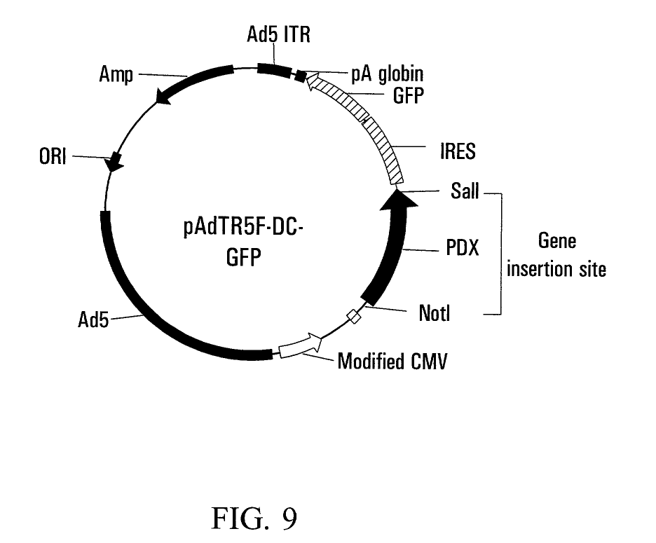

Recombinant adenoviruses, were constructed using replication-defective adenoviral vectors deleted of sequences spanning E1A, E1B, and a portion of the E3 region, impairing the ability of the virus to replicate (reference 42). The gene encoding full length PDX mRNA was inserted into the multiple cloning site of the previously described (reference 43) transfer vector pAd-TR5F-DC-GFP (FIG. 9) and placed under the control of a modified CMV promoter containing a tetracycline (tet)-regulated expression cassette (reference 44) and expressed together with the green fluorescent protein (GFP) tracer. Recombinant adenoviruses were constructed by in vitro homologous recombination in 293 cells as described (reference 45). Recombinant viruses were amplified and purified from cell lysates by cesium chloride gradient ultracentrifugation followed by desalting on a Sephadex G-50 column.

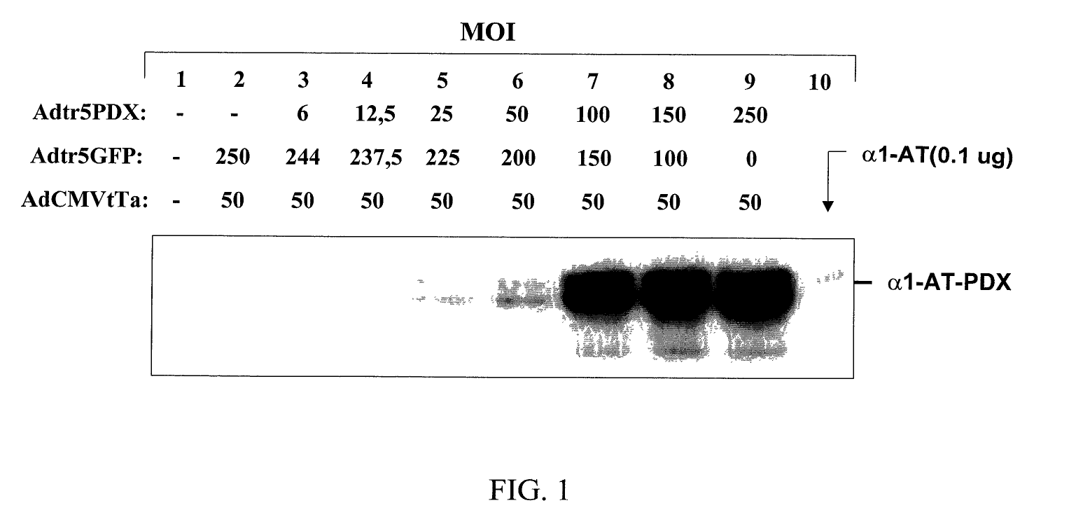

To test the ability of AdTR5-PDX vector to direct the expression of PDX, rat synovial cells were either non-infected or infected in hybridoma serum-free medium (Gibco BRL) at various multiplicity of infection (MOI) with Adtr5PDX and/or Adtr5GFP in the presence of AdCMVtTA (encoding the transactivator tTA) used at a MOI of 50. For each sample, the MOI of Adtr5GFP was adjusted for a final MOI of 300. Following 48 h infection, cell lysates are prepared with 1% NP-40-containing lysis buffer supplemented with Complete Protease Inhibitor Cocktail (Roche Diagnostics GmbH, Mannheim, Germany). Fifteen μg per sample are then resolved into reducing 10% SDS-PAGE gels. Separated proteins are then transferred onto nitrocellulose membranes, blocked for 1 h, and probed overnight with affinity-purified anti-human α₁ Antitrypsine IgG (1:500) (ICN BIOMEDICALS, Costa Mesa, Calif.). Immunoreactive bands are revealed using the ECL detection system (Amhersham Canada Limited, Oakville, ON). Upon infection with adenovirus-encoding PDX, rat synoviocyte cells produce the 55 kDa immunoreactive PDX that increases with the multiplicity of infection of the PDX adenoviral vector (see FIG. 1, lanes 3-9). This band has the same migration pattern as pure recombinant human α₁-antitrypsin (lane 10).

### EXAMPLE 2

Regulatable Production of PDX

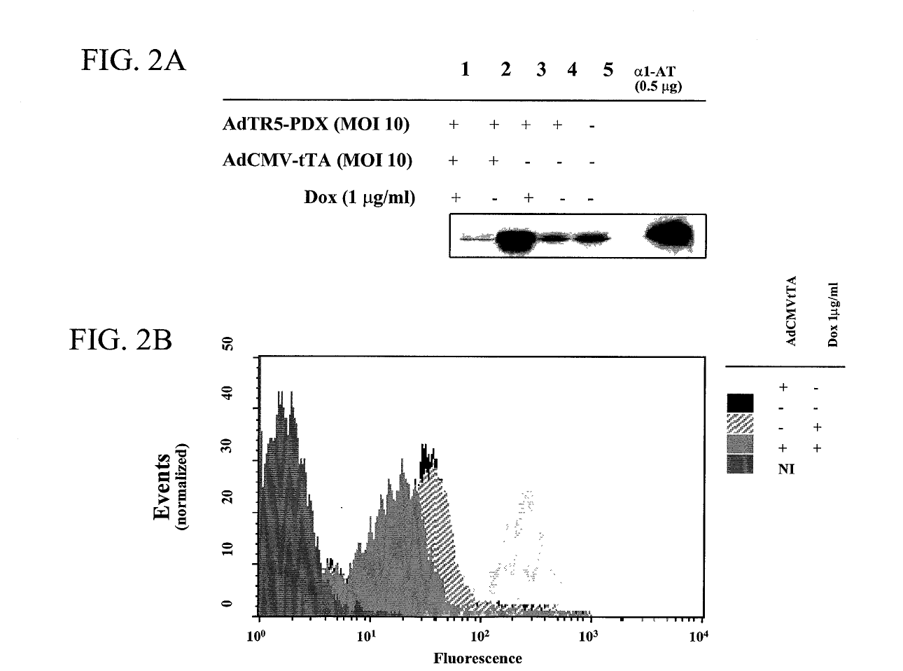

The transduction system used in Example 1 was used to test the ability of AdTR5-PDX to mediate regulatable transgene expression in 293 cells. The cells were infected with AdTR5-PDX and/or AdCMVtTA in the presence or absence of doxycycline (Dox) (1 μg/ml) and 24 h after infection, cell cultures were assessed for both A) PDX production by Western blotting as described in Example 1 and B) GFP fluorescence. For fluorescence analysis, cells were pelleted, resuspended in PBS, and fixed with paraformaldehyde (2% final) for 30 min at 4° C. GFP emission was evaluated using a FACScan flow cytometer (BECTON DICKINSON, San Jose, Calif.) equipped with an argon-ion laser and configured for analysis of fluoresceine. Results were analyzed using CellQuest™ software (BECTON DICKINSON). Results in FIG. 2 indicated that the addition of 1 μg/ml Dox to the cultures abolished PDX production induced by the transactivator tTa (lane 1 compared to lane 2) without affecting basal levels of PDX (lane 4 compared to lane 3). Parallel results were observed when cells are analyzed for fluorescence intensity (FIG. 2B).

### EXAMPLE 3

Inhibition of Furin-Mediated Processing of Human Transforming Growth Factor β1 by PDX

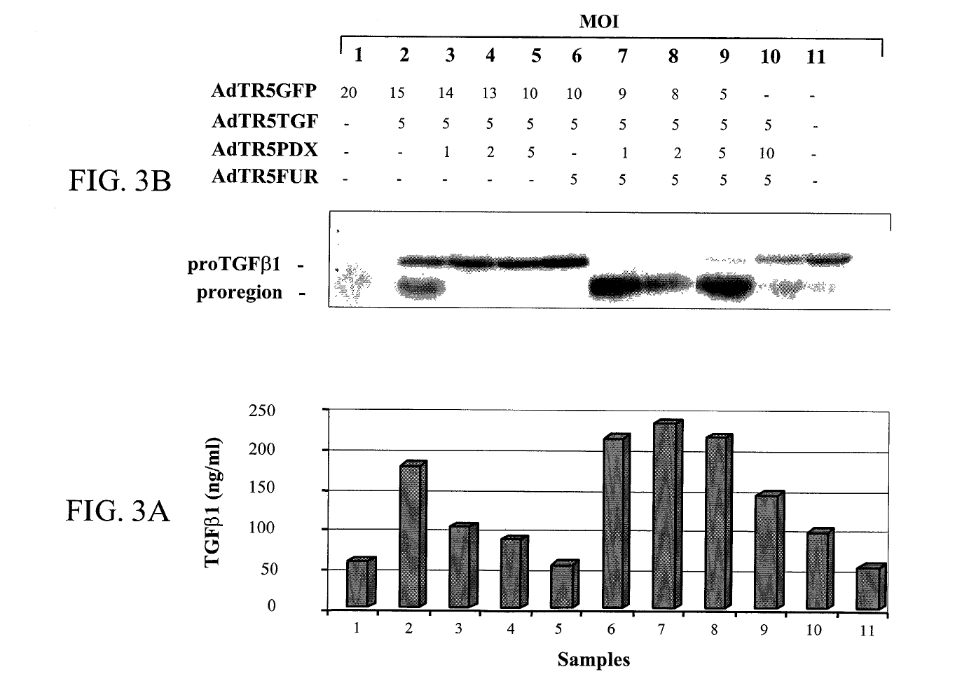

Synovial cells were infected with AdTR5-PDX (encoding PDX) and/or AdTR5-TGF (encoding human TGF β1 precursor) and/or AdTR5FUR (encoding human furin) and/or control AdTR5GFP. Forty-eight hours after infection, concentrated cell supernatants were assessed by Western blotting for A) the production of proteolytic fragments in immunoblotting using TGF β1-specific anti-LAP antibodies (R&D Systems, Minneapolis, Minn.) and B) the production of bioactive TGF β1 using a TGF β1-specific ELISA assay (R&D Systems, Minneapolis, Minn.). Co-infection of cells with AdTR5-PDX abrogated pro-TGF β1 proteolytic processing mediated by endogenous cellular enzyme(s) (FIG. 3A, lanes 3-5) while co-expression with the control virus (AdTR5GFP) did not affect basal level of TGF β proteolytic processing (FIG. 3A, lane 2). As a control, co-infection of synovial cells with AdTR5FUR, encoding furin, resulted in complete processing of TGF β1 precursor (FIG. 3A, lane 6) which is also inhibited by PDX co-infection (FIG. 3A, lanes 7-10). In parallel, the amounts of active TGF β1 released in cell culture medium were diminished by the expression of α₁-PDX (FIG. 3B).

### EXAMPLE 4

PDX Inhibits Furin-Mediated Production of the Endogenous Mature Form of TGF β1

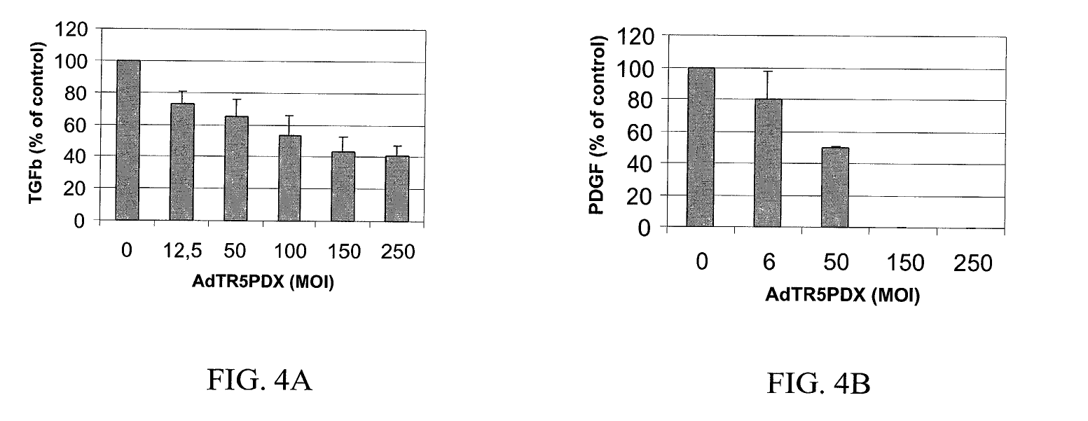

In parallel experiments, applicant tested the ability of AdTR5-PDX to inhibit the endogenous production of mature TGF β1 by synovial cells. The cells were infected as described in Example 1 with the MOI of Adtr5PDX compensated with the control Adtr5GFP for a final MOI of 300. After 48 h incubation, the supernatants from infected cells were assessed for their release of TGF β1 as described in Example 3. Infection of cells with 0 to 250 MOI of AdTR5-PDX reduces the amounts of active TGF β1 released in cell culture (FIG. 4A) with 60% inhibition observed at a MOI of 250. The results demonstrated in Examples 3 and 4 indicate that PDX is capable of inhibiting endoprotease-mediated processing of TGF β1 precursor in vivo and immediately suggested a method for treating TGF β-related pathological conditions by inhibiting the furin convertase.

### EXAMPLE 5

PDX Inhibits Furin-Mediated Production of the Endogenous Mature Form of PDGF

Applicant also tested the ability of AdTR5-PDX to inhibit the endogenous production of mature PDGF by synovial cells. Supernatant from infected cells were assessed for their release of PDGF in supernatants using a PDGF-specific ELISA assay (R&D Systems, Minneapolis, Minn.). Cells were infected as described in Example 1. Infection of cells with 0 to 250 MOI of AdTR5-PDX reduces the amounts of active TGF β1 released in cell culture (FIG. 4B) with 100% inhibition observed at a MOI of 150 and 250. For each sample, the MOI of Adtr5PDX was compensated with the control Adtr5GFP for a final MOI of 300. The results demonstrated that PDX is capable of inhibiting endoprotease-mediated production of the mature form of PDGF and suggested a novel method for treating PDGF-related inflammatory/growth conditions by inhibiting the furin convertase.

### EXAMPLE 6

Inhibition of Furin-Mediated Processing of Human TACE by PDX

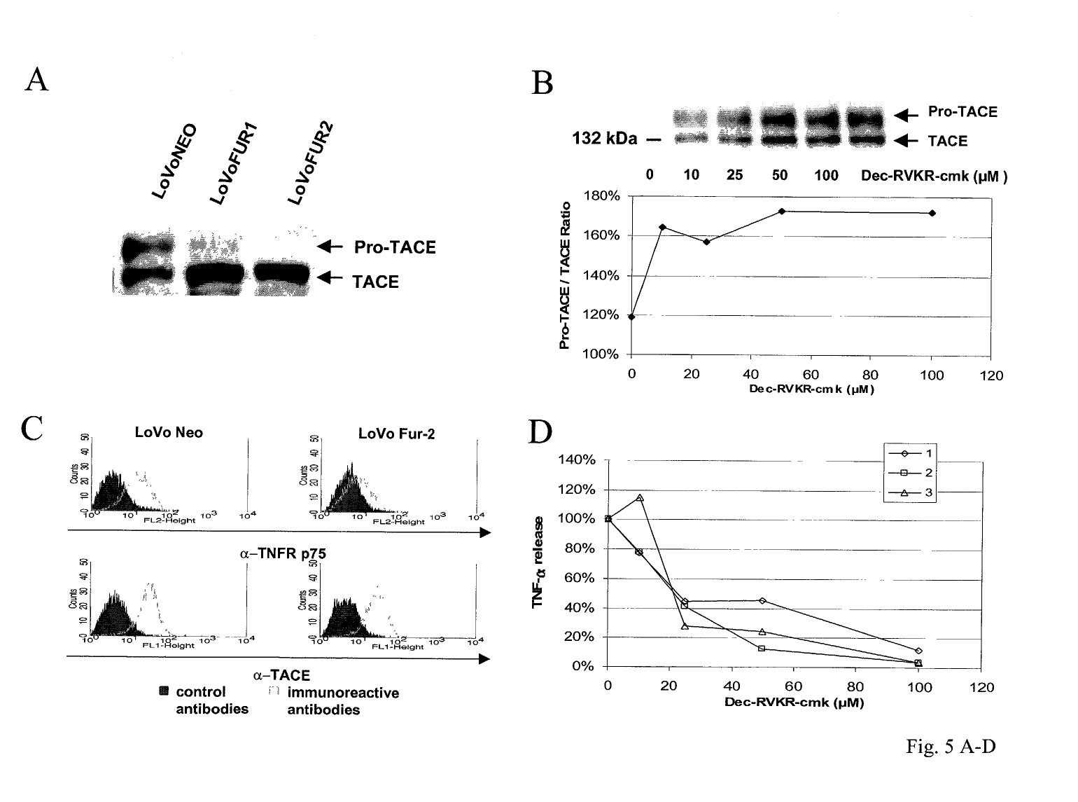

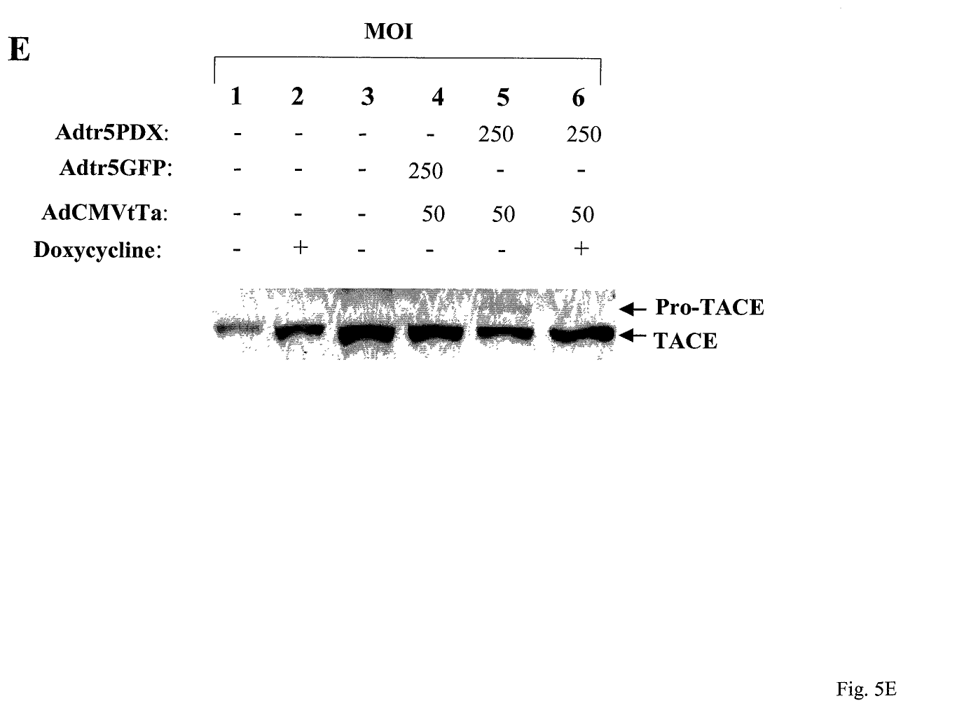

To assess the involvement of furin in TACE endoproteolytic processing, applicant first used a furin-deficient cell line, the LoVo cells. These are human colon carcinoma cells which have a point mutation in both alleles of the fur gene leading to production of a defective enzyme (reference 46). These furin knock-out cells have been extensively used to study the contribution of furin in a cellular context (references 10, 38 and 47). LoVo cells were transfected with wild type furin and two stable transfectants LoVoFUR1 and LoVoFUR2 and control transfectant LoVoNEO have been generated accordingly to their acquired gentamycin resistance. Cell lysates were assessed by Western blotting for the production of TACE proteolytic fragments in immunoblotting using TACE-specific antibodies (Chemicon International, CA). As shown in FIG. 5A, LoVoNEO cells expressed about 50% of processed TACE as seen by the relative intensity of the mature TACE and the proTACE immunoreactive bands. Genetic complementation with furin increases their endogenous production of the mature form of TACE with complete processing observed with the LoVo FUR2 clone indicating the requirement of intact furin for optimal processing of the TACE precursor molecule.

To define if proprotein convertases other than furin are involved in TACE maturation, LoVo NEO cells were incubated for 24 hrs in the presence of Dec-RVKR-CH₂Cl, a synthetic peptide that mimics proprotein recognition site. Such inhibitor has been shown to efficiently inhibit the enzymatic activity of most members of the proprotein convertases including furin, PC6B, PC3, PC2, PACE-4 and PC7 (48). LoVo NEO cells were incubated for 24 hrs in the presence of various concentrations of Dec-RVKR-CH₂Cl, and cell lysates were assessed for TACE maturation by Western blotting as described above. As shown in FIG. 5B, the addition of Dec-RVKR-CH₂Cl to LoVo NEO cells results in a further inhibition of TACE maturation, clearly indicating that other proprotein convertases family members than furin are involved in TACE maturation.

TACE has been shown to mediate cleavage of TNFα as well as a variety of ectodomain including the TNF p75 receptor (49). To investigate whether the levels of TACE maturation observed in LoVo transfectants impacts TACE-related activities, we first measured cell-surface p75 TNF receptor expression as a marker for TACE cell-surface activity. Briefly, LoVo NEO and LoVo FUR2 cell samples were labeled with anti-TACE or anti-TNF p75 receptor antibodies (Catlag laboratories, Burlingame, Calif.) and cell fluorescence was analyzed on a FACScan (Becton Dickinson). Results expressed in FIG. 5C, indicated that furine complementation of LoVo cells reduces cell-surface p75 TNF receptor expression (48% reduction) witout affecting the levels of cell surface TACE. Next, we measured the ability of Dec-RVKR-CH₂Cl to block TNFα release from MonoMac-1 cells, a human monocytic cell line known to produce high levels of this cytokine (50). For this, MonoMac-1 cells were preincubated for 22 hrs with varying concentrations of Dec-RVKR-CH₂Cl, then 500 ng/ml LPS and 100 ng/ml PMA were added. After 3 hours incubation with PMA and LPS, the supernatants were tested for TNF-α production using a TNF-α-specific ELISA assay (R&D Systems, Minneapolis, Minn.). Results expressed in FIG. 5D indicated that treatment of these cells with a proprotein convertase inhibitor blocked almost completely TNFα released with an ED 50 obtained around 20 μM. These results suggest that inhibition of TACE processing leads to an impairment in TACE activities.

Applicant also tested the ability of AdTR5-PDX to inhibit the endogenous production of mature TACE by synovial cells. Synovial cells were infected with AdTR5PDX and/or control AdTR5GFP and stimulated or not with mouse TNF α (20 ng/ml). Forty-eight hours after infection, cell lysates were assessed by Western blotting for the production of TACE proteolytic fragments in immunoblotting using TACE-specific antibodies (Chemicon International, CA). Control synovial cells produce only the mature form of TACE (FIG. 5E, lane 1). Forty-eight hours of stimulation of synovial cells with TNF α resulted in an increase in the immunoreactive band corresponding to the mature form of TACE (lanes 2 to 6). Infection of cells with AdTR5-PDX resulted in the production of an additional immunoreactive TACE band corresponding to the TACE precursor form (proTACE) indicating that PDX can reduce TACE processing mediated by endogenous furin enzyme. As an additional control, the addition of 1 μg/ml Dox to the cultures abolished PDX-mediated block in TACE processing.

### EXAMPLE 7

Inhibition of Furin-Mediated Processing of Endogenous Gelatinase A (MMP-2) by PDX

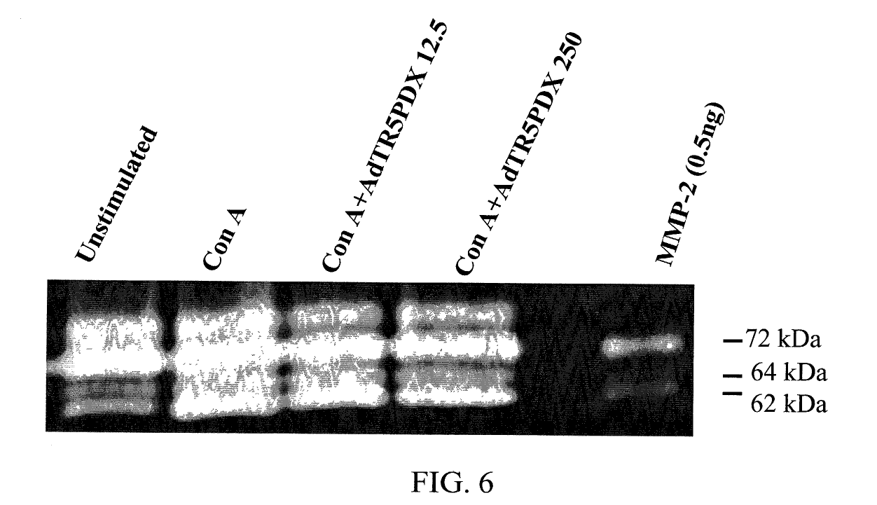

Gelatinolytic activity in the harvested culture media was detected by gelatin zymography with 10% (w/v) acrylamide gel containing gelatin (0.6 mg/ml; Difco) as described previously (reference 51). When rat synoviocytes were treated with Con A (10 μg/ml), a spontaneously produced 72 kDa pro-MMP-2 was converted into a 62 kDa active MMP-2 with the appearance of a 64 kDa intermediate form (FIG. 6). Infection of cells with AdTR5-PDX inhibited the augmentation of pro-MMP-2 activation in Con A-treated rat synoviocytes in a MOI-dependent manner. These results demonstrated that PDX is capable of inhibiting endoprotease-mediated processing of gelatinase A precursor in vivo and suggested a method to inhibit gelatinase A mediated conjunctive tissue degradation by inhibiting the furin convertase.

### EXAMPLE 8

Inhibition of Synovial Cell Growth by PDX

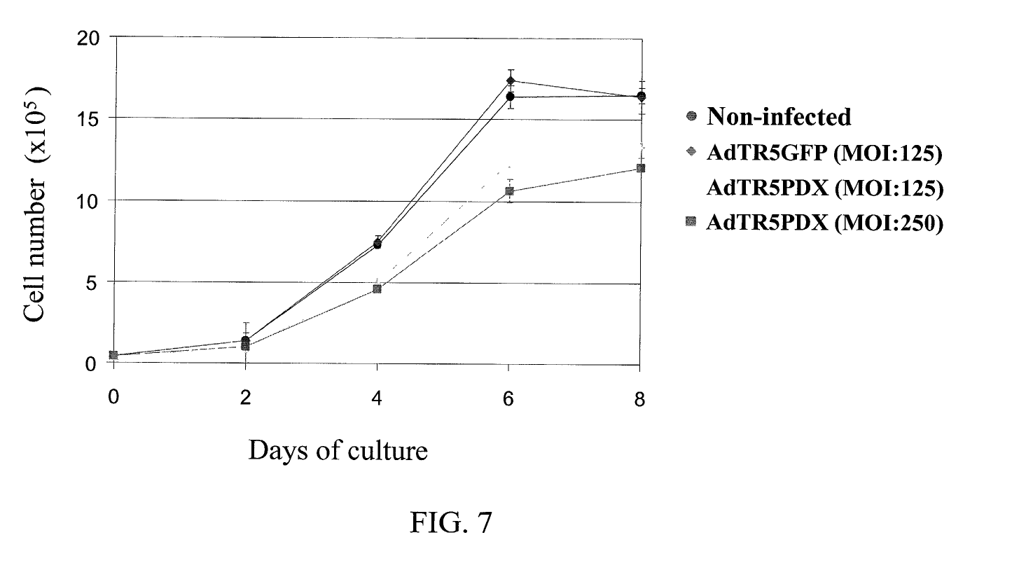

Effect of AdTR5-PDX on cell proliferation was evaluated by growth curve. Rat synovial cells (5×104 cells per well) seeded in culture plates were either non-infected or infected with control virus Adtr5GFP or Adtr5PDX virus. On days 0, 2, 4, 6, and 8, cells were recovered by trypsin-EDTA treatment, and their number was counted using an hematocytometer under the microscope. The proliferation rate of AdTR5-PDX-infected cells was significantly reduced compared with cells infected with control AdTR5GFP (FIG. 7). Therefore, inhibition of endogenous furin proteases results in reduction of the proliferation capacity of synovial cells. These results demonstrated that PDX, through inhibition of endogenous furin convertase, has novel growth inhibitory properties and suggested a method for treating growth-related pathological conditions in which the furin convertase is involved.

### EXAMPLE 9

Inhibition of Collagen-Induced Arthritis (CIA) by PDX

Collagen-induced arthritis (CIA) is induced in female Lewis rats by i.d. administration of heterologous (bovine) type II collagen (250 μg) solubilized in 0.1M acetic acid and emulsified in Freund's Complete Adjuvant (FCA) while control groups receive FCA only (day 0). Synovitis typically develops 12-15 days postimmunization in 80-90% of the animals. The severity of the arthritis was evaluated using an established macroscopic scoring system. A score of 1 is given for isolated ankle involvement; 2, for the ankle and the proximal ½ of the tarsal joint; 3, for the ankle and the entire tarsal joint down to the metatarsal joints, and 4, for the involvement of the entire paw, inducing the digits. The sum of the scores for each paws is calculated as the arthritic index. Therapeutic effects of virus injection were examined by arthritis score and paw tickness measured using a caliper.

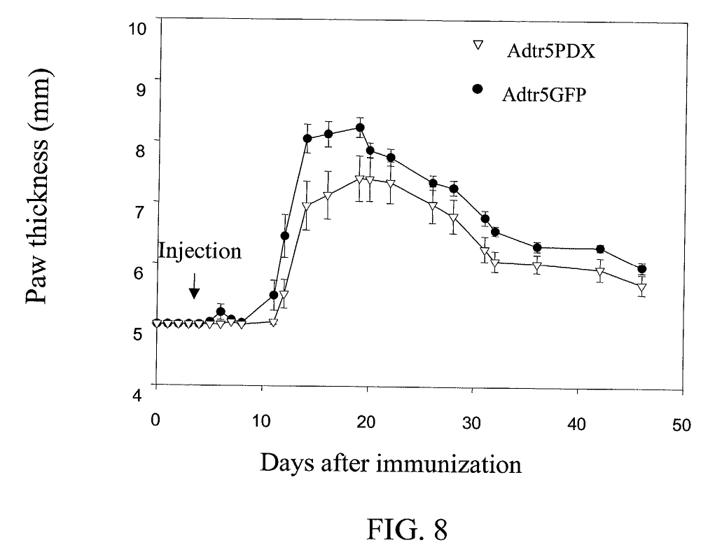

For treatment, stocks of recombinant virus purified on cesium chloride gradient are used for intraarticular injection. The induction of CIA and monitoring of the disease is performed essentially as described above. Five days after CIA induction, animals were randomly distributed into two groups and AdTRr5PDX (3×10⁸ pfu in 12 μl buffer) resuspended in Tris (pH 7.4), 1 mM MgCl₂ and 10% (v/v) glycerol was infused into the right ankle using sterile technique and a medial approach (N=8). The left ankle was injected with (3×10⁸ pfu) control AdTR5GFP adenovirus (n=8). The data represented in FIG. 8 indicates that injection of Adtr5PDX results in a significant (p<0.05, N=8) reduction of ankle joint inflammation especially during the acute phase (days 12-20) of the disease with mean GFP-treated group measure of 7.74+/−0.14 mm compared to 6.87+/−0.18 mm for the group injected with control adenovirus ((p<0.05, N=40). Parallel results were obtained with the direct measurement of arthritic index for days 11-20) where applicant observed a mean group index of 3.0+/−0.10 for control adenovirus compared with 2.25+/−0.15 (p<0.05 N=88) for Adtr5PDX injected ankles. Therefore, PDX, through inhibition of endogenous furin convertase, has novel in vivo anti-inflammatory and anti arthritic properties suggesting a method for treating inflammatory-related pathological conditions in which the furin convertase is involved.

### EXAMPLE 10

Involvement of the Convertase Furin in Aggrecanase Naturation/Activation

To define the role of furin in the activation aggrecanase-1, aggrecanase-1 cDNAs are cloned in an appropriate mammalian expression vector. The generated recombinant proteins are C-terminal His or V5 tagged for convenient detection of the precursor and C-terminal mature forms in Western blotting. Parental and FUR-1 and FUR-2 LoVo cells as described in Example 6 are transfected with the aggrecanase-1 expressing vector.

The influence of furin expression in metalloproteinase fragmentation is monitored. LoVo cell lysates are analyzed for production of aggrecanase-1-related digestion products by Western blot using anti-His or V5 antibodies. Using this system, a ˜77 kDa immunoreactive bands represents the unprocessed recombinant aggrecanase whereas the immunoreactive processed forms are revealed at ˜64 kDa.

To ensure that furin conversion results in the enzymatically active form of the metalloproteinases, results from Western blots are corroborated with the measure of aggrecanase enzymatic activity. For aggrecanase-1 activity, concentrated medium is incubated in the presence of purified aggrecan substrate and the generated aggrecan fragments evaluated in Western blots using the anti-NITEGE antisera (BC-3 antibody). This antisera reveals a neoepitope generated by cleavage of aggrecan between Glu³⁷³ and Ala³⁷⁴ sequence (references 51 and 52). In this way, BC-3 immunoreactive bands are detected ranging in molecular mass from approximately 250 to 40 kDa. As a control, there is used the BC-14 antibody that reacts with the neoepitope generated by cleavage between the aggrecanase unrelated site Asn³⁴¹ and Phe³⁴².

### REFERENCES

1. Van de Ven, W. J., Roebroek, A. J., and Van Duijnhoven, H. L. 1993. Structure and function of eukaryotic proprotein processing enzymes of the subtilisin family of serine proteases. J. Crit. Rev. Onc. 4:115-136.

2. Seidah, N. G., Hamelin, J., Mamarbachi, M., Dong, W., Tardos, H., Mbikay, M., Chretien, M., and Day, R. 1996. cDNA structure, tissue distribution, and chromosomal localization of rat PC7, a novel mammalian proprotein convertase closest to yeast kexin-like proteinases. Proc. Natl. Acad. Sci. USA. 93:3388-3393.

3. Molloy, S. S., Thomas, L., VanSlyke, J. K., Stenberg, P. E., and Thomas, G. 1994. Intracellular trafficking and activation of the furin proprotein convertase: localization to the TGN and recycling from the cell surface. EMBO J. 13:18-33.

4. Molloy, S. S., Anderson, E. D., Jean, F., and Thomas, G. 1999. Bi-cycling the furin pathway: from TGN localization to pathogen activation and embryogenesis. Trends Cell. Biol. 9:28-35.

5. Takahashi, S., Hatsuzawa, K., Watanabe, T., Murakami, K., and Nakayama, K. 1994. Sequence requirements for endoproteolytic processing of precursor proteins by furin: transfection and in vitro experiments. J. Biochem. (Tokyo) 116:47-52.

6. Betsholtz C, Johnsson A, Heldin C H, Westermark B, Lind P, Urdea M S, Eddy R, Shows T B, Philpott K, Mellor A L and et al. 1986. cDNA sequence and chromosomal localization of human platelet-derived growth factor A-chain and its expression in tumour cell lines Nature 320 (6064), 695-699.

7. Collins T, Ginsburg D, Boss J M, Orkin S H, Pober J S 1985. Cultured human endothelial cells express platelet-derived growth factor B chain: cDNA cloning and structural analysis. Nature 316(6030):748-50.

8. Kingsle D. M. 1994. The TGF-beta superfamily: new members, new receptors, and new genetic tests of function in different organisms. Genes Dev. 8(2):133-46.

9. Bergeron F, Leduc R, Day R. 2000. Subtilase-like pro-protein convertases: from molecular specificity to therapeutic applications. J Mol Endocrinol. 2000, 24(1):1-22.

10. Dubois, C. M., Laprise, M. H., Blanchette, F., Gentry, L. E. and Leduc, R. 1995. Processing of transforming growth factor β1 precursor by human furin convertase. J. Biol. Chem. 270:10618-24.

11. Keyszer, G. M., Heer, A. H., and Gay, S. 1994. Cytokines and oncogenes in cellular interactions of rheumatoid arthritis. Stem Cells. 12:75-86.

12. Remmers, E. F., Sano, H., and Wilder, R. L. 1991. Platelet-derived growth factors and heparin-binding (fibroblast) growth factors in the synovial tissue pathology of rheumatoid arthritis. Sem. Arthritis Rheum. 21:191-199.

13. Butler, D. M., Leizer, T., and Hamilton, J. A. 1989. Stimulation of human synovial fibroblast DNA synthesis by platelet-derived growth factor and fibroblast growth factor. Differences to the activation by IL-1. J. Immunol. 142:3098-103.

14. Smith, R. J., Justen, J. M., Sam, L M., Rohloff, N. A., Ruppel, P. L., Brunden, M. N., and Chin, J. E. 1991. Platelet-derived growth factor potentiates cellular responses of articular chondrocytes to interleukin-1. Arthritis Rheum. 34:697-706.

15. Wilder, R. L., Case, J. P., Crofford, L. J., Kumkumian, G. K., Lafyatis, R., Remmers, E. F., Sano, H., Sternberg, EM., and Yocum, D. E. 1991. Endothelial cells and the pathogenesis of rheumatoid arthritis in humans and streptococcal cell wall arthritis in Lewis rats. J. Cell. Biochem. 45:162-166.

16. Reibman, J., Meixler, S., Lee, T. C., Gold, L. I., Cronstein, B. N., Haines, K. A., Kolasinski, S. L., and Weissmann, G. 1991. Transforming growth factor β1, a potent chemoattractant for human neutrophils, bypasses classic signal-transduction pathways. Proc. Natl. Acad. Sci. USA 88:6805-6809.

17. Brandes, M. E., Mai, U. E., Ohura, K., and Wahl, S. M. 1991. Type I transforming growth factor-β receptors on neutrophils mediate chemotaxis to transforming growth factor-beta. J. Immunol. 147:1600-1606.

18. Wahl, S. M., Hunt, D. A., Wakefield, L. M., McCartney-Francis, N., Wahl, L. M., Roberts, A. B. and Sporn, M. B. 1987. Transforming growth factor type β induces monocyte chemotaxis and growth factor production. Proc. Natl. Acad. Sci. USA 84:5788-5792.

19. McCartney,-Francis, N., Mizel, D., Wong, H., Wahl, L., and Wahl, S. 1990. TGF-β regulates production of growth factors and TGF-β by human peripheral blood monocytes. Growth Factors. 4:27-35.

20. Wahl S M. Allen J B. Costa G L. Wong H L. Dasch J R. 1993. Reversal of acute and chronic synovial inflammation by anti-transforming growth factor β. J. Exp. Med. 177:225-30.

21. Brandes, M. E., Allen, J. B., Ogawa, Y., and Wahl, S. M. 1991. Transforming growth factor β1 suppresses acute and chronic arthritis in experimental animals. J. Clin. Invest. 87:1108-1113.

22. Black, R. A. et al. 1997. A metalloproteinase disintegrin that releases tumour-necrosis factor-α from cells. Nature 385:729-731.

23. Roghani, M., Becherer, J. D., Moss, M. L., Atherton, R. E., Erdjument-Bromage, H., Arribas, J., Blackburn, R. K., Weskamp, G., Tempst, P., and Blobel, C. P. 1999. Metalloprotease-disintegrin MDC9: intracellular maturation and catalytic activity. J. Biol. Chem. 274:3531-3540.

24. Tortorella M D, Burn T C, Pratta M A, Abbaszade I, Hollis J M, Liu R, Rosenfeld S A, Copeland R A, Decicco C P, Wynn R, Rockwell A, Yang F, Duke J L, Solomon K, George H, Bruckner R, Nagase H, Itoh Y, Ellis D M, Ross H, Wiswall B H, Murphy K, Hillman M C Jr, Hollis G F, Arner E C, et al. 1999. Protein, Nucleotide Purification and cloning of aggrecanase-1: a member of the ADAMTS family of proteins. Science. 284(5420):1664-1666.

25. Abbaszade I., Liu R. Q., Yang F., Rosenfeld S. A., Ross O. H., Link J. R., Ellis D. M., Tortorella M. D., Pratta M. A., Hollis J. M., Wynn R., Duke J. L., George H. J., Hillman M. C. Jr, Murphy K., Wiswall B. H., Copeland R. A., Decicco C. P., Bruckner R., Nagase H., Itoh Y., Newton R. C., Magolda R. L., Trzaskos J. M., Burn T. C., et al. Cloning and characterization of ADAMTS11, an aggrecanase from the ADAMTS family. J Biol Chem. 13;274(33):23443-23450

26. Massova I., Kotra L. P., Fridman R., Mobashery S. 1998. Matrix metalloproteinases: structures, evolution, and diversification. FASEB J. 12(12):1075-1095.

27. Pei D. Identification and characterization of the fifth membrane-type matrix metalloproteinase MT5-MMP. 1999. J Biol Chem. 26;274(13):8925-8932.

28. Schlondorff J, Becherer J D, Blobel C P. 2000. Intracellular maturation and localization of the tumour necrosis factor alpha convertase (TACE). Biochem J. 2000 347:131-138.

29. Clarke H R, Wolfson M F, Rauch C T, Castner B J, Huang C P, Gerhart M J, Johnson R S, Cerretti D P, Paxton R J, Price V L, Black R A. 1998. Expression and purification of correctly processed, active human TACE catalytic domain in Saccharomyces cerevisiae. Protein Expr Purif. 13(1):104-10.

30. Maquoi E, Noel A, Frankenne F, Angliker H, Murphy G, Foidart J M. 1998. Inhibition of matrix metalloproteinase 2 maturation and HT1080 invasiveness by a synthetic furin inhibitor. FEBS Lett. 13;424(3):262-266.

31. Sato T, Kondo T, Fujisawa T, Seiki M, Ito A. 1999. Furin-independent pathway of membrane type 1-matrix metalloproteinase activation in rabbit dermal fibroblasts. J Biol Chem. 274(52):37280-37284.

32. Paleolog, E. 1997. Target effector role of vascular endothelium in the inflammatory response: insights from the clinical trial of anti-TNF alpha antibody in rheumatoid arthritis. Mol. Pathol. 50:225-233.

33. Moreland, L. W., Baumgartner, S. W., Schiff, M. H., Tindall, E. A., Fleischmann, R. M., Weaver, A. L., Ettlinger, R. E., Cohen, S., Koopman, W. J., Mohler, D., Widmer, M. B., and Blosch, C. M. 1997. Treatment of rheumatoid arthritis with a recombinant human tumor necrosis factor receptor (p75)-Fc fusion protein. N. Engl. J. Med. 337:141-147.

34. Ohshima, S. Saeki, Y. Mima, T., Sasai, M., Nishioka, K., Ishida, H., Shimizu, M., Suemura, M., McCloskey, R., and Kishimoto, T. 1999. Long-term follow-up of the changes in circulating cytokines, soluble cytokine receptors, and white blood cell subset counts in patients with rheumatoid arthritis (RA) after monoclonal anti-TNF α antibody therapy. J. Clin. Immunol. 19:305-313.

35. Konttinen Y. T., Ceponis A., Takagi M., Ainola M., Sorsa T., Sutinen M., Salo T., Ma J., Santavirta S., Seiki M. 1998. New collagenolytic enzymes/cascade identified at the pannus-hard tissue junction in rheumatoid arthritis: destruction from above. Matrix Biol. 17(8-9):585-601.

36. Little, C. B., Flannery, C. R., Hughes, C. E., Mort, J. S., Roughley, P. J., Dent. C., and Caterson, B. 1999. Aggrecanase versus matrix metalloproteinases in the catabolism of the interglobular domain of aggrecan in vitro. Biochem. J. 344:61-68.

37. Anderson E. D., Thomas L., Hayflick J. S., Thomas G. 1993. Inhibition of HIV-1 gp160-dependent membrane fusion by a furin-directed α1-antitrypsin variant. J. Biol. Chem. 268(33):24887-91.

38. Pei D. Weiss S. J. 1995. Furin-dependent intracellular activation of the human stromelysin-3 zymogen. Nature. 375(6528):244-7.

39. Jean, F., Stella, K., Thomas, L., Liu, G., Xiang, Y., Reason, A. J., and Thomas, G. 1998. α1-Antitrypsin Portland, a bioengineered serpin highly selective for furin: application as an antipathogenic agent. Proc. Natl. Acad. Sci. USA 95:7293-7298.

40. Cui, Y., Jean, F., Thomas, G., and Chrisian, J. L. 1998. BMP-4 is proteolytically activated by furin and/or PC6 during vertebrate embryonic development. EMBO J. 17:4735-4743.

41. Watanabe M., Hirano A., Stenglein S., Nelson J., Thomas G., Wong T. C. 1995. Engineered serine protease inhibitor prevents furin-catalyzed activation of the fusion glycoprotein and production of infectious measles virus. J. Virol. 69(5):3206-10.

42. Hurwitz, D. R., and Chinnadurai, G. 1985. Evidence that a second tumor antigen coded by adenovirus early gene region E1a is required for efficient cell transformation. Proc. Natl. Acad. Sci. USA. 82:163-167.

43. Mosser D. D., Caron A. W., Bourget L., Jolicoeur P., Massie B. 1997. Use of a dicistronic expression cassette encoding the green fluorescent protein for the screening and selection of cells expressing inducible gene products. Biotechniques 22:150-4, 156, 158-61.

44. Manfred, G., and Bujard, H. 1992. Tight control of gene expression in mammalian cells by tetracycline-responsive promoters. Proc. Natl. Acad. Sci. USA 89:5547-5551.

45. Petrof, B. J., Acsadi, G., Jani, A., Massie, B., Bourdon, J., Matusiewicz, N., Yang, L. Lochmuller, H., and Karpati, G. 1995. Efficiency and functional consequences of adenovirus-mediated in vivo gene transfer to normal and dystrophic (mdx) mouse diaphragm. Am. J. Resp. Cell & Mol. Biol. 13:508-517.

46. Takahashi, S., Nakagawa, T., Kasai, K., Banno, T., Duguay, S. J., Van de Ven, W. J., Murakami, K., and Nakayama, K. 1995. A second mutant allele of furin in the processing-incompetent cell line, LoVo. Evidence for involvement of the homo B domain in autocatalytic activation. J. Biol. Chem. 270:26565-26569.

47. Hallenberger, S., Bosch, V., Angliker, H., Shaw, E., Klenk, H. D., and Garten, W. 1992. Inhibition of furin-mediated cleavage activation of HIV-1 glycoprotein gp160. Nature. 360:358-361.

48. Jean F., Stella K., Thomas L., Liu G., Xiang Y., Reason A. J., Thomas G. 1998. alpha1-Antitrypsin Portland, a bioengineered serpin highly selective for furin: application as an antipathogenic agent. Proc Natl Acad Sci USA 23: 7293-8.

49. Peschon J J, Slack J L, Reddy P, Stocking K L, Sunnarborg S W, Lee D C, Russell W E, Castner B J, Johnson R S, Fitzner J N, Boyce R W, Nelson N, Kozlosky C J, Wolfson M F, Rauch C T, Cerretti D P, Paxton R J, March C J, Black R A. 1998. An essential role for ectodomain shedding in mammalian development. Science. 282:1281-4.

50. Steube K. G., Teepe D., Meyer C., Zaborski M., Drexler H. G. 1997. A model system in haematology and immunology: the human monocytic cell line MONO-MAC-1. Leuk Res. 21:327-35.

51. Lark, M. W., et al. 1995. Quantification of a matrix metalloproteinase-geneated aggrecan G1 fragment using monospecific and anti-peptide serum. Biochem J. 307:245-252.

52. Lark, M. W. et al. 1995. Cell-mediated catabolism of aggrecan. Evidence that cleavage at the aggrecanase site (Glu373-Ala374) is a primary event in proteolysis of the interglobular domain. J. Biol. Chem. 270:2550-2556.

The disclosures of all the above-mentioned references are herein incorporated by reference.

1. A method for the treatment in a mammal of an inflammatory or erosive disease, said method comprising administering to the mammal a compound capable of inhibiting a proprotein convertase.

2. The method of claim 1, wherein the inflammatory or erosive disease is characterized by furin or furin-like protease activity.

3. The method of claim 1, wherein the compound is selected from PDX or a construct, variant, analog, PDX-related peptide, PDX-related-peptidomimetic, their salts, complexes or derivatives.

4. The method of claim 2, wherein the compound is selected from PDX or a construct, variant, analog, PDX-related peptide, PDX-related-peptidomimetic, their salts, complexes or derivatives.

5. The method of claim 1 wherein the compound is administered in the form of a prodrug.

6. The method of claim 1 wherein the compound is administered in combination with an intracellular carrier.

7. The method of claim 1 wherein the compound is administered by a gene therapy delivery system.

8. The method of claim 7 wherein a cell transfectant is used as the gene therapy delivery system.

9. The method of claim 1 wherein proprotein convertase-mediated endoproteolytic activation of TACE in cells of the mammal are blocked.

10. The method of claim 9 wherein the proprotein convertase-mediated endoproteolytic activation is furin-mediated endoproteolytic activation.

11. The method of claim 1 wherein proprotein convertase-mediated endoproteolytic activation of TGFβ in cells of the mammal are blocked.

12. The method of claim 11 wherein the proprotein convertase-mediated endoproteolytic activation is furin-mediated endoproteolytic activation.

13. The method of claim 1 wherein proprotein convertase-mediated endoproteolytic activation of mature PDGF in cells of the mammal are blocked.

14. The method of claim 13 wherein the proprotein convertase-mediated endoproteolytic activation is furin-mediated endoproteolytic activation.

15. The method of claim 1 wherein proprotein convertase-mediated growth in cells of the mammal are blocked.

16. The method of claim 15 wherein the proprotein convertase-mediated growth is furin-mediated growth.

17. The method of claim 1 wherein proprotein convertase-mediated endoproteolytic activation of aggrecanase-1 in cells of the mammal are blocked.

18. The method of claim 17 wherein the proprotein convertase-mediated endoproteolytic activation is furin-mediated endoproteolytic activation.

19. The method of claim 1 wherein the inflammatory disease is rheumatoid arthritis.

20. A composition for treatment in a mammal of an inflammatory or erosive disease, said composition comprising a compound selected from PDX or a construct, variant, analog, PDX-related peptide, PDX-related-peptidomimetic, their salts, complexes or derivatives.


SEQUENCE LISTING (3 sequences)

SEQ ID NO: 1 - protein, 4 aa, Homo sapiens
  SITE (1)..(4): Reactive site of alpha1-antitrypsin
AIPM

SEQ ID NO: 2 - protein, 4 aa, Artificial Sequence
  Variant of Homo sapiens alpha1-antitrypsin reactive site
RIPR

SEQ ID NO: 3 - protein, 4 aa, Artificial Sequence
  Synthetic peptide
RVKR